SEQUENCE LISTING (2 sequences)

SEQ ID NO: 1 - DNA, 20 bp, Artificial sequence
  Synthetic oligonucleotide
agcttcttgtccagctttat

SEQ ID NO: 2 - DNA, 20 bp, Artificial sequence
  Synthetic oligonucleotide
tcttggttacatgaaatccc

(19) United States
(12) Patent Application Publication
(10) Pub. No.: US 2024/0092822 A1
(43) Pub. Date: Mar. 21, 2024

(54) PURIFICATION METHODS FOR OLIGOMERIC COMPOUNDS

(71) Applicant: Ionis Pharmaceuticals, Inc., Carlsbad, CA (US)

(72) Inventors: Andrew A. Rodriguez, San Diego, CA (US); Christopher Michael Gabriel, Oceanside, CA (US)

(73) Assignee: Ionis Pharmaceuticals, Inc., Carlsbad, CA (US)

(21) Appl. No.: 18/548,711

(22) PCT Filed: Mar. 2, 2022
(86) PCT No.: PCT/US2022/018447

§ 371 Date: Sep. 1, 2023

Related U.S. Application Data
(60) Provisional application No. 63/155,515, filed on Mar. 2, 2021.

(51) Int. Cl. C07H 21/02; B01D 15/16; B01D 15/20; B01D 15/32; B01D 15/42

(52) U.S. Cl. CPC C07H 21/02; B01D 15/166; B01D 15/203; B01D 15/325; B01D 15/426

(57) ABSTRACT

The present disclosure provides a process of separating designated oligomeric compounds from sample solutions comprising at least one contaminant In certain embodiments, the designated oligomeric compounds comprise a modified oligonucleotide and a conjugate group comprising at least one carbohydrate cluster. In certain embodiments, the present disclosure provides HPLC conditions that increase the separation of a designated oligomeric compound from at least one contaminant compared to standard HPLC conditions.

### SEQUENCE LISTING

The present application is being filed along with a Sequence Listing in electronic format. The Sequence Listing is provided as a file entitled DVCM0047WOSEQ_ST25.txt created Feb. 14, 2022, which is 1 kb in size. The information in the electronic format of the sequence listing is incorporated herein by reference in its entirety.

### FIELD

The present disclosure provides methods for separating a designated oligomeric compound having at least one conjugate group comprising a carbohydrate cluster from contaminants in solution.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics, or affinity for a target nucleic acid. Conjugate groups may be attached to an antisense compound to enhance one or more properties, such as pharmacokinetics, pharmacodynamics, and uptake into cells and/or tissues of interest.

Oligomeric compounds comprising an oligonucleotide and at least one conjugate group are chemically synthesized in a multi-step process that has the potential to introduce a number of unwanted contaminants. The separation of desired oligomeric compounds from such contaminants remains an important challenge.

### SUMMARY

The present disclosure provides an improved process of separating designated oligomeric compounds from sample solutions comprising at least one contaminant. In certain embodiments, the designated oligomeric compounds comprise a modified oligonucleotide and a conjugate group comprising at least one carbohydrate cluster. In certain embodiments, such contaminants arise during the synthesis of oligomeric compounds. In certain embodiments, such contaminants are modified oligonucleotides lacking a conjugate group.

The present disclosure provides the following non-limiting embodiments:

Embodiment 1. A process for separating a designated oligomeric compound from at least one contaminant in sample solution,
wherein the designated oligomeric compound comprises a modified oligonucleotide and a conjugate group having at least one carbohydrate moiety, comprising:
a) Providing an HPLC column packed with an HPLC stationary phase,
b) Adding equilibration solution to the HPLC column to equilibrate the column,
c) Adding the designated oligomeric compound and at least one contaminant in sample solution onto the equilibrated HPLC column,
d) Adding equilibration solution to the HPLC column,
e) Optionally, washing the HPLC column with washing solution,
f) Adding elution solution to the HPLC column to elute the oligomeric compound from the column, and collecting the eluate in separate tubes at various time points, and
   thereby separating the designated oligomeric compound from the contaminant;
   wherein:
   the equilibration solution comprises 0% to 20% organic solvent, 50 mM to 500 mM salt, and 50 to 200 mM of a boronic acid, and has a pH of 7.0 or greater;
   the washing solution comprises 0% to 50% of the same organic solvent as the equilibration solution, and 50 to 500 mM salt, and has a pH of 10 or greater; and
   the elution solution comprises 50% to 90% of the same organic solvent as is in the equilibration solution, 50 to 500 mM salt, and 0-200 mM of a boronic acid, and has a pH of 7.0 or greater.
   Embodiment 2. The process of embodiment 1, wherein at least one column volume of equilibration solution is added to the HPLC column in step (d).
   Embodiment 3. The process of embodiment 1 or 2 that does not include washing step (e).
   Embodiment 4. The process of embodiment 1 or 2 comprising the washing step (e).
   Embodiment 5. The process of embodiment 4, wherein at least 2 column volumes of washing solution are added.
   Embodiment 6. The process of any of embodiments 1-5, wherein at least one column volume of elution solution is added at step (f).
   Embodiment 7. The process of embodiment 1, wherein the boronic acid of the equilibration solution is selected from phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, 4-methoxyphenylboronic acid, boric acid, methylboronic acid, propylboronic acid, butylboronic acid, and pentylboronic acid.
   Embodiment 8. The process of embodiment 7, wherein the boronic acid is an aryl boronic acid.
   Embodiment 9. The process of embodiment 7, wherein the aryl boronic acid is selected from phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, and 4-methoxyphenylboronic acid.
   Embodiment 10. The process of embodiment 7, wherein the boronic acid is selected from butyl boronic acid, propyl boronic acid, and pentyl boronic acid.
   Embodiment 11. The process of any of embodiments 1-10, wherein the equilibration solution comprises 50 mM of the boronic acid.
   Embodiment 12. The process of any of embodiments 1-10, wherein the equilibration solution comprises 100 mM of the boronic acid.
   Embodiment 13. The process of any of embodiments 1-10, wherein the equilibration solution comprises 200 mM of the boronic acid.
   Embodiment 14. The process of any of embodiments 1-13, wherein the pH of the equilibration solution is from 7 to 7.5.
   Embodiment 15. The process of any of embodiments 1-13, wherein the pH of the equilibration solution is from 7.5 to 8.5.
   Embodiment 16. The process of any of embodiments 1-13, wherein the pH of the equilibration solution is from 8.5 to 9.5.
   Embodiment 17. The process of any of embodiments 1-13, wherein the pH of the equilibration solution is from 9.5 to 10.5.
   Embodiment 18. The process of any of embodiments 1-13, wherein the pH of the equilibration solution is from 10.5 to 11.5.
   Embodiment 19. The process of any of embodiments 1-13, wherein the pH of the equilibration solution is from 11.5 to 12.5.
   Embodiment 20. The process of any of embodiments 1-19, wherein the elution solution contains 50 mM of the boronic acid.
   Embodiment 21. The process of any of embodiments 1-19, wherein the elution solution contains 100 mM of the boronic acid.
   Embodiment 22. The process of any of embodiments 1-19, wherein the elution solution contains 150 mM of the boronic acid.
   Embodiment 23. The process of any of embodiments 1-19, wherein the elution solution contains 200 mM of the boronic acid.
   Embodiment 24. The process of any of embodiments 1-23, wherein the pH of the elution solution is from 7 to 7.5.
   Embodiment 25. The process of any of embodiments 1-23, wherein the pH of the elution solution is from 7.5 to 8.5.
   Embodiment 26. The process of any of embodiments 1-23, wherein the pH of the elution solution is from 8.5 to 9.5.
   Embodiment 27. The process of any of embodiments 1-23, wherein the pH of the elution solution is from 9.5 to 10.5.
   Embodiment 28. The process of any of embodiments 1-23, wherein the pH of the elution solution is from 10.5 to 11.5.
   Embodiment 29. The process of any of embodiments 1-23, wherein the pH of the elution solution is from 11.5 to 12.5.
   Embodiment 30. The process of any of embodiments 1-29, wherein the equilibration solution and the elution solution have the same concentration of boronic acid.
   Embodiment 31. The process of any of embodiments 1-30, wherein the equilibration solution and the elution solution have the same pH.
   Embodiment 32. The process of any of embodiments 1-31, wherein the equilibration solution and the elution solution are the same other than the organic solvent:water ratio.
   Embodiment 33. The process of any of embodiments 1-32, wherein the equilibration solution contains 3-8%, 8-12%, 12-18%, 18-22%, 22-27%, or 27-33% organic solvent.
   Embodiment 34. The process of any of embodiments 1-33, wherein the elution solution contains 55-65%, 65-75%, or 75-85% organic solvent.
   Embodiment 35. The process of embodiment 33 or 34, wherein the equilibration solution comprises 10% organic solvent and the elution solution contains 70% organic solvent.
   Embodiment 36. The process of any of embodiments 4-35, wherein the washing solution contains 3-8%, 8-12%, or 12-18% organic solvent.
   Embodiment 37. The process of any of embodiments 1-36, wherein the organic solvent is selected from acetonitrile, methanol, isopropanol, ethanol, and tetrahydrofuran.
   Embodiment 38. The process of embodiment 37, wherein the organic solvent is methanol.
   Embodiment 39. The process of any of embodiments 1-38, wherein the equilibration solution and the elution solution contain 150-250 mM salt.
   Embodiment 40. The process of embodiments 39, wherein the salt of the equilibration solution and the elution solution is, independently, sodium acetate, sodium chloride, sodium carbonate, or sodium phosphate.
   Embodiment 41. The process of embodiment 40, wherein the salt is sodium acetate.
   Embodiment 42. The process of any of embodiments 1-41, wherein the elution solution is added in a step gradient.
   Embodiment 43. The process of any of embodiments 1-42, wherein the elution solution is added in a linear gradient.
   Embodiment 44. The process of any of embodiments 1-2 or 4-43, wherein the washing solution is added in a step gradient.
   Embodiment 45. The process of any of embodiments 1-2 or 4-43, wherein the washing solution is added in a linear gradient.
   Embodiment 46. The process of any of embodiments 1-45, wherein the HPLC stationary phase is selected from polystyrene, surface-modified polystyrene, surface-modified silica, and surface-modified methylacrylate.
   Embodiment 47. The process of any of embodiments 1-46, wherein the HPLC stationary phase is monodisperse polystyrene.
   Embodiment 48. The process of any of embodiments 1-46, wherein the HPLC stationary phase is surface-modified and the surface-modification comprises a boronic acid functionality.
   Embodiment 49. The process of any of embodiments 1-48, wherein the designated oligomeric compound comprises a modified oligonucleotide consisting of 10-30 linked nucleosides.
   Embodiment 50. The process of any of embodiments 1-49, wherein the designated oligomeric compound comprises a conjugate group having at least one GalNAc sugar.
   Embodiment 51. The process of embodiment 50, wherein the conjugate group comprises a triantennary GalNAc cell-targeting moiety.
   Embodiment 52. The process of embodiment 51, wherein the conjugate group comprises the structure:

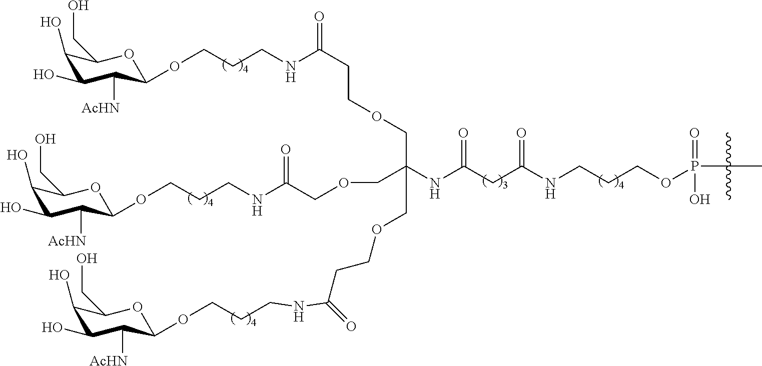

Embodiment 53. The process of any of embodiments 1-52, wherein at least one contaminant is an unconjugated modified oligonucleotide consisting of 10-30 linked nucleosides.
Embodiment 54. The process of embodiment 53, wherein the unconjugated modified oligonucleotide has the same nucleobase sequence, internucleoside linkage chemistry, and sugar motif as the modified oligonucleotide of the designated oligomeric compound.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase.

As used herein, “HPLC stationary phase” means a stationary phase used in high performance liquid chromatography. In certain embodiments, HPLC stationary phase is contained within an HPLC column. In certain embodiments, HPLC stationary phase is commercially available in pre-packed HPLC columns. In certain embodiments, HPLC stationary phase is composed of polystyrene. In certain embodiments, HPLC stationary phase is monodisperse polystyrene. In certain embodiments, HPLC stationary phase is surface-modified. In certain embodiments, HPLC stationary phase is surface-modified silica, methyl acrylate, or polystyrene. In certain embodiments, HPLC stationary phase is C18 surface-modified silica.

As used herein, “mobile phase” means the solution that flows over an HPLC stationary phase. In certain embodiments, “mobile phase” is equilibration solution. In certain embodiments, “mobile phase” is washing solution. In certain embodiments, “mobile phase” is elution solution.

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-modified” in reference to a furanosyl sugar moiety or nucleoside comprising a furanosyl sugar moiety means the furanosyl sugar moiety comprises a substituent other than H or OH at the 2′-position of the furanosyl sugar moiety. 2′-modified furanosyl sugar moieties include non-bicyclic and bicyclic sugar moieties.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified furanosyl sugar moiety. In certain embodiments, the bicyclic sugar moiety does not comprise a furanosyl moiety.

As used herein, “cEt” means a 4′ to 2′ bridge in place of the 2′-OH-group of a ribosyl sugar moiety, wherein the bridge has the formula of 4′-CH(CH3)-O-2′, and wherein the methyl group of the bridge is in the S configuration. A “cEt sugar moiety” is a bicyclic sugar moiety with a 4′ to 2′ bridge in place of the 2′-OH-group of a ribosyl sugar moiety, wherein the bridge has the formula of 4′-CH(CH3)-O-2′, and wherein the methyl group of the bridge is in the S configuration. “cEt” means constrained ethyl. As used herein, “cEt nucleoside” means a nucleoside comprising a cEt sugar moiety.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “cell-targeting moiety” means a conjugate group or portion of a conjugate group that is capable of binding to a particular cell type or particular cell types.

As used herein, “gapmer” means an oligonucleotide or a portion of an oligonucleotide having a central region comprising a plurality of nucleosides that support RNase H cleavage positioned between a 5′-region and a 3′-region. Herein, the 3′- and 5′-most nucleosides of the central region each comprise a 2′-deoxyfuranosyl sugar moiety. Herein, the 3′-most nucleoside of the 5′-region comprises a 2′-modified sugar moiety or a sugar surrogate. Herein, the 5′-most nucleoside of the 3′-region comprises a 2′-modified sugar moiety or a sugar surrogate. The “central region” may be referred to as a “gap”; and the “5′-region” and “3′-region” may be referred to as “wings”.

As used herein, “internucleoside linkage” means a group or bond that forms a covalent linkage between adjacent nucleosides in an oligonucleotide. As used herein “modified internucleoside linkage” means any internucleoside linkage other than a naturally occurring, phosphodiester internucleoside linkage. Modified internucleoside linkages may or may not contain a phosphorus atom. A “neutral internucleoside linkage” is a modified internucleoside linkage that is mostly or completely uncharged at pH 7.4 and/or has a pKa below 7.4.

As used herein, “non-bicyclic sugar” or “non-bicyclic sugar moiety” means a sugar moiety that comprises fewer than 2 rings. Substituents of modified, non-bicyclic sugar moieties do not form a bridge between two atoms of the sugar moiety to form a second ring.

As used herein, “linked nucleosides” are nucleosides that are connected in a continuous sequence (i.e. no additional nucleosides are present between those that are linked).

As used herein, “2′-MOE” means a 2′-OCH₂CH₂OCH₃ group in place of the 2′-OH group of a furanosyl sugar moiety. A “2′-MOE sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl. As used herein, “2′-MOE nucleoside” means a nucleoside comprising a 2′-MOE sugar moiety.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 2-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, “sugar moiety” means an unmodified sugar moiety or a modified sugar moiety. As used herein, “unmodified sugar moiety” means a P-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not have a stereoconfiguration other than β-D-ribosyl. Modified furanosyl sugar moieties include bicyclic sugars and non-bicyclic sugars.

As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, if a solution has 0% of a particular component, it means that there is none of that component in that solution.

As used herein, “carbohydrate” means a monosaccharide, disaccharide, trisaccharide, tetrasaccharide, oligosaccharide, polysaccharide, or derivatives thereof. In certain embodiments, a carbohydrate is N-acetylgalactosamine.

As used herein, “GalNAc” means a conjugated N-acetyl galactosamine, an amino sugar derivative of galactose, represented by the structure below:

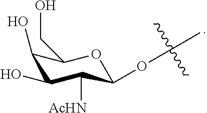

### Certain Process for the Purification of Oligonucleotides

The present disclosure provides an improved process of purifying oligomeric compounds from a mixture comprising at least one contaminant. In certain embodiments, the oligomeric compounds comprise a modified oligonucleotide and a conjugate group comprising at least one carbohydrate cluster. In certain embodiments, contaminants arise during the synthesis of oligomeric compounds. In certain embodiments, such contaminants are modified oligonucleotides lacking a conjugate group. In certain embodiments, such contaminants are oligomeric compounds lacking a carbohydrate cluster.

The present disclosure provides HPLC purification conditions that achieve improved separation compared to standard HPLC purification conditions for oligomeric compounds.

### Certain HPLC Purification Conditions for Oligomeric Compounds

High-performance liquid chromatography (HPLC) is a process of separating organic molecules by flowing a sample mixture in a equilibration solution over a column containing a solid adsorbent material (HPLC stationary phase), followed by changing the solution flowing over the column in order to elute the material that was adsorbed onto the stationary phase. Typically, the first mobile phase is an equilibration solution that contains low organic solvent, and the mobile phase used to elute the material that is adsorbed onto the stationary phase, or elution solution, contains high organic solvent. In certain embodiments, a linear gradient is used to gradually change the ratio of two or more solutions flowing over the column. In certain embodiments, a step gradient is used to rapidly change the ratio of two or more solutions flowing over the stationary phase. As the amount of organic solvent increases, the adsorbed organic molecules are eluted from the HPLC column and can be collected in eluent fractions. In certain embodiments, three or more HPLC solution are used. In certain such embodiments, a washing solution is added to the HPLC column after the equilibration solution and before the elution solution.

The present disclosure provides an improved process for HPLC separation of oligomeric compounds comprising conjugate groups having at least one carbohydrate moiety from contaminants in solution. Increased separation is observed between oligomeric compounds comprising carbohydrate-containing conjugates and unconjugated modified oligonucleotides when a boronic acid is included in the HPLC buffer. Improved separation is observed when the boronic acid is included in the equilibration solution, the elution solution, or both the equilibration solution and the elution solution. In certain embodiments, a washing solution is also used. In certain such embodiments, a boronic acid is included in only the equilibration solution. In certain embodiments, the boronic acid is phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, 4-methoxyphenylboronic acid, boric acid, methylboronic acid, propylboronic acid, butylboronic acid, or pentylboronic acid. In certain embodiments, the boronic acid is an aryl boronic acid. In certain embodiments, the boronic acid is phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, or 4-methoxyphenylboronic acid. In certain embodiments, each of the equilibration solution, the washing solution, and the elution solution is at a specific pH. In certain embodiments, each mobile phase is at pH 7-7.5, 7.5-8.5, 8.5-9.5, 9.5-10.5, 10.5-11.5, 11.5-12.5, 7, 8, 9, 10, 11, or 12. In certain embodiments, the equilibration solution and the elution solution are at the same pH as each other. In certain such embodiments, the equilibration solution and the elution solution are at pH 7-7.5, 7.5-8.5, 8.5-9.5, 9.5-10.5, 10.5-11.5, 11.5-12.5, 7, 8, 9, 10, 11, or 12. In certain embodiments, the pH of the washing solution is greater than 10. In certain embodiments, each mobile phase comprises an organic solvent. In certain embodiments, only the elution solution comprises an organic solvent. In certain embodiments, only the washing solution and the elution solution comprise an organic solvent. In certain embodiments, the organic solvent is present in the equilibration solution at less than 5%, less than 10%, less than 15%, less than 20%, 5%-10%, 10%-15%, 15%-20%, or 10-20% by volume. In certain embodiments, the organic solvent is present in the elution solution at greater than 30%, greater than 40%, greater than 50%, greater than 60%, greater than 70%, 30-100%, 30-35%, 35-40%, 40-45%, 45-50%, 50%-100%, 50%-55%, 55%-60%, 60%-65%, 65%-70%, 70%-75%, 68%-72%, 75%-80%, 80%-85%, 85%-90%, or 90-95% by volume. In certain embodiments, the organic solvent is methanol, acetonitrile, isopropanol, ethanol, or tetrahydrofuran. In certain embodiments, the organic solvent is methanol. In certain embodiments, the equilibration solution, the washing solution, and/or the elution solution comprise a salt. In certain embodiments, the salt is present in the equilibration solution, the washing solution, and/or the elution solution at 50 mM-100 mM, 100-150 mM, 150-200 mM, 175-225 mM, or 200-250 mM. In certain embodiments, the salt is selected from sodium acetate, sodium chloride, sodium carbonate, and sodium phosphate. In certain embodiments, the salt is sodium acetate.

In certain embodiments, processes described herein are useful for purifying mixtures containing oligomeric compounds comprising oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage). The present disclosure provides processes of purifying oligomeric compounds comprising oligonucleotides that have any number of modifications described herein.

### I. Modifications

### A. Modified Nucleosides

In certain embodiments, synthetic processes described herein are used to produce compounds comprising modified nucleosides comprising a modified sugar moiety, a modified nucleobase, or both a modified sugar moiety and a modified nucleobase. Certain such compounds are described.

### 1. Certain Modified Sugar Moieties

In certain embodiments, sugar moieties are non-bicyclic, modified furanosyl sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or non-bicyclic 2′-modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂ N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH₃, and OCH₂CH₂OCH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′- CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b}, is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b}, is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al.,; Wengel eta., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. 6,794,499; Wengel et al., U.S. Pat. No. 6,670,461; Wengel et al., U.S. Pat. No.7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191;; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

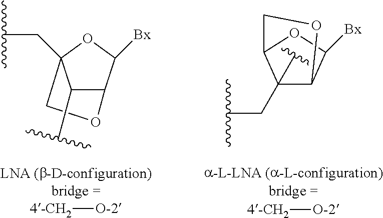

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as “2′-modified”, indicates that the sugar moiety comprises the indicated modification at the 2′ position and may comprise additional modifications and/or substituents. The term “substituted” following a position of the furanosyl ring, such as “2′-substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by formula I:

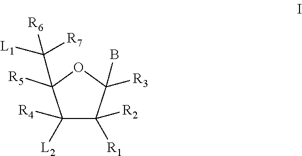

wherein B is a nucleobase; and L₁ and L₂ are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R_{1-4, 6, 7} are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted furanosyl sugar moiety is represented by formula I, wherein B is a nucleobase; and L₁ and L₂ are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by formula II:

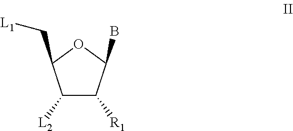

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by formula III:

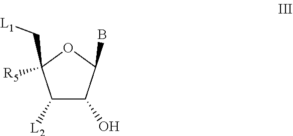

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by formula IV:

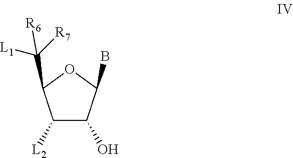

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by formula V:

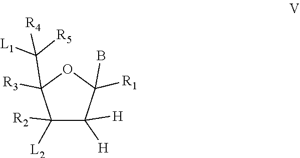

wherein B is a nucleobase; and L₁ and L₂ are each, independently, an internucleoside linkage, a terminal group, a conjugate group, or a hydroxyl group. Each of R₁₋₅ are independently selected from H and a non-H substituent. If all of R₁₋₅ are each H, the sugar moiety is an unsubstituted 2′-deoxyfuranosyl sugar moiety. The stereochemistry is not defined unless otherwise noted.

In the context of a nucleoside and/or an oligonucleotide, a 4′-substituted 2′-deoxyribosyl sugar moiety is represented by formula VI:

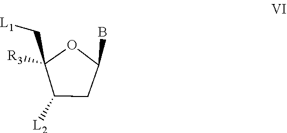

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by formula VII:

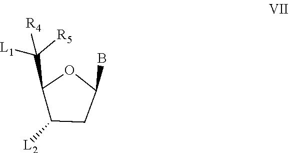

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include (β-L-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by formula VIII:

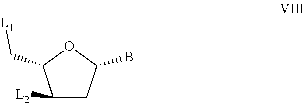

wherein B is a nucleobase; and L₁ and L₂ are each, independently, an internucleoside linkage, a terminal group, a conjugate group, or a hydroxyl group. The stereochemistry is defined as shown.

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

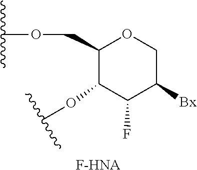

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

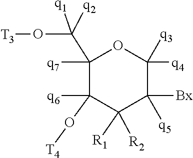

wherein, independently, for each of said modified THP nucleoside:

Bx is a nucleobase moiety;

T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

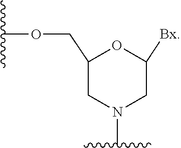

In certain embodiments, morpholinos may be modified, for example by adding or altering various substituent groups from the above morpholino structure. Such sugar surrogates are refered to herein as “modified morpholinos.”

Many other bicyclic and tricyclic sugar and sugar surrogate ring systems are known in the art that can be used in modified nucleosides.

### 2. Modified Nucleobases

In certain embodiments, synthetic processes disclosed herein are useful for making oligomeric compounds having at least one modified nucleoside comprising a modified nucleobase. Modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In certain embodiments, processes described herein are useful for synthesizing compounds that comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

In certain embodiments, processes described herein are useful for synthesizing oligomeric compounds having one or more modified internucleoside linkage. In certain embodiments, such compounds are selected over compounds having only phosphate diester internucleoside linkages because of desirable properties such as, for example, enhanced cellular uptake, enhanced affinity for target nucleic acids, and increased stability in the presence of nucleases.

The two main classes of internucleoside linkages are defined by the presence or absence of a phosphorus atom. Representative phosphorus-containing internucleoside linkages include unmodified phosphate diester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, and phosphorodithioate (“HS-P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioate diesters. Modified oligonucleotides comprising internucleoside linkages having a chiral center can be prepared as populations of modified oligonucleotides comprising stereorandom internucleoside linkages, or as populations of modified oligonucleotides comprising phosphorothioate diester linkages in particular stereochemical configurations. In certain embodiments, populations of modified oligonucleotides comprise phosphorothioate diester internucleoside linkages wherein all of the phosphorothioate diester internucleoside linkages are stereorandom. Such modified oligonucleotides can be generated using synthetic methods that result in random selection of the stereochemical configuration of each phosphorothioate diester linkage. Nonetheless, as is well understood by those of skill in the art, each individual phosphorothioate diester of each individual oligonucleotide molecule has a defined stereoconfiguration. In certain embodiments, populations of modified oligonucleotides are enriched for modified oligonucleotides comprising one or more particular phosphorothioate diester internucleoside linkages in a particular, independently selected stereochemical configuration. In certain embodiments, the particular configuration of the particular phosphorothioate diester linkage is present in at least 65% of the molecules in the population. In certain embodiments, the particular configuration of the particular phosphorothioate diester linkage is present in at least 70% of the molecules in the population. In certain embodiments, the particular configuration of the particular phosphorothioate diester linkage is present in at least 80% of the molecules in the population. In certain embodiments, the particular configuration of the particular phosphorothioate diester linkage is present in at least 90% of the molecules in the population. In certain embodiments, the particular configuration of the particular phosphorothioate diester linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate iester in the (Sp) configuration. In certain embodiments, a population of modified oligonucleotides is enriched for modified oligonucleotides having at least one phosphorothioate diester in the (Rp) configuration. In certain embodiments, modified oligonucleotides comprising (Rp) and/or (Sp) phosphorothioate diesters comprise one or more of the following formulas, respectively, wherein “B” indicates a nucleobase:

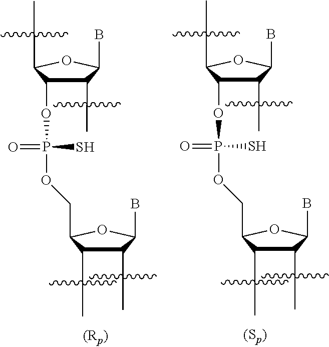

Unless otherwise indicated, chiral internucleoside linkages of modified oligonucleotides described herein can be stereorandom or in a particular stereochemical configuration. The oxidizing agents described herein are suitable for use in synthesis of oligonucleotides having one or more chirally controlled linkage.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, 0, S and CH2 component parts.

In certain embodiments, synthetic processes disclosed herein result in a phosphate diester internucleoside linkage. Nevertheless, in certain embodiments, other internucleoside linkages within an oligonucleotide or oligomeric compound may be any of the linkages described above.

### II. Certain Motifs

In certain embodiments, synthetic processes described herein are useful for making oligomeric compounds having any motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, synthetic processes described herein are useful for making oligonucleotides that comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, synthetic processes described herein are useful for making modified oligonucleotides that comprise or have a uniformly modified sugar motif. An oligonucleotide comprising a uniformly modified sugar motif comprises a segment of linked nucleosides, wherein each nucleoside of the segment comprises the same modified sugar moiety. An oligonucleotide having a uniformly modified sugar motif throughout the entirety of the oligonucleotide comprises only nucleosides comprising the same modified sugar moiety. For example, each nucleoside of a 2′-MOE uniformly modified oligonucleotide comprises a 2′-MOE modified sugar moiety. An oligonucleotide comprising or having a uniformly modified sugar motif can have any nucleobase sequence and any internucleoside linkage motif.

### B. Certain Nucleobase Motifs

In certain embodiments, synthetic processes described herein are useful for making oligonucleotides that comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### C. Certain Internucleoside Linkage Motifs

The synthetic processes described herein are particularly useful in synthesizing oligonucleotides or oligomeric compounds having particular linkage motifs. In certain embodiments, oligonucleotides comprise modified and/or unmodified internucleoside linkages arranged along the oligonucleotide or region thereof in a defined pattern or motif. In certain embodiments, each internucleoside linkage of a modified oligonucleotide is a phosphorothioate diester internucleoside linkage (P═S) and the compound includes a conjugate group comprising at least one phosphate diester. In certain embodiments, each internucleoside linkage of a modified oligonucleotide is independently selected from a phosphorothioate diester internucleoside linkage and phosphate diester internucleoside linkage. In certain embodiments, each phosphorothioate diester internucleoside linkage is independently selected from a stereorandom phosphorothioate diester, a (Sp) phosphorothioate diester, and a (Rp) phosphorothioate diester. In certain embodiments, the terminal internucleoside linkages are modified. In certain embodiments, the internucleoside linkage motif comprises at least one phosphate diester internucleoside linkage in at least one of the 5′-region and the 3′-region, wherein the at least one phosphate diester linkage is not a terminal internucleoside linkage, and the remaining internucleoside linkages are phosphorothioate diester internucleoside linkages. In certain such embodiments, all of the phosphorothioate diester linkages are stereorandom. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate diester internucleoside linkages. In certain embodiments, all of the internucleoside linkages of the oligonucleotide are phosphorothioate diester internucleoside linkages. In certain embodiments, each internucleoside linkage of the oligonucleotide is selected from phosphate diester or phosphate and phosphorothioate diester and at least one internucleoside linkage is a phosphorothioate diester and at least one internucleoside linkage is a phosphate diester.

In certain embodiments, the oligonucleotide comprises at least 6 phosphorothioate diester internucleoside linkages. In certain embodiments, the oligonucleotide comprises at least 8 phosphorothioate diester internucleoside linkages. In certain embodiments, the oligonucleotide comprises at least 10 phosphorothioate diester internucleoside linkages. In certain embodiments, the oligonucleotide comprises at least one block of at least 6 consecutive phosphorothioate diester internucleoside linkages. In certain embodiments, the oligonucleotide comprises at least one block of at least 8 consecutive phosphorothioate diester internucleoside linkages. In certain embodiments, the oligonucleotide comprises at least one block of at least 10 consecutive phosphorothioate diester internucleoside linkages. In certain embodiments, the oligonucleotide comprises at least block of at least one 12 consecutive phosphorothioate diester internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, it is desirable to arrange the number of phosphorothioate diester internucleoside linkages and phosphate diester internucleoside linkages to maintain nuclease resistance. In certain embodiments, it is desirable to arrange the number and position of phosphorothioate diester internucleoside linkages and the number and position of phosphate diester internucleoside linkages to maintain nuclease resistance. In certain embodiments, the number of phosphorothioate diester internucleoside linkages may be decreased and the number of phosphate diester internucleoside linkages may be increased. In certain embodiments, the number of phosphorothioate diester internucleoside linkages may be decreased and the number of phosphate diester internucleoside linkages may be increased while still maintaining nuclease resistance. In certain embodiments it is desirable to decrease the number of phosphorothioate diester internucleoside linkages while retaining nuclease resistance. In certain embodiments it is desirable to increase the number of phosphate diester internucleoside linkages while retaining nuclease resistance.

### III. Certain Modified Oligonucleotides

In certain embodiments, oligonucleotides synthesized using processes described herein consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides synthesized using processes described herein have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### IV. Certain Conjugated Compounds

In certain embodiments, the oligomeric compounds synthesized using processes described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides synthesized using processes described herein are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937),₌a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., W02014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the synthetic processes described herein are useful for making conjugate linkers comprising one or more phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphate diester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphate diester, a phosphate ester, a carbamate, or a disulfide. In certain embodiments, a cleavable bond is one or both of the esters of a phosphate diester. In certain embodiments, a cleavable moiety comprises a phosphate or phosphate diester. In certain embodiments, the cleavable moiety is a phosphate linkage between an oligonucleotide and a conjugate moiety or conjugate group. In certain embodiments, the synthetic processes described herein are useful for making phosphate diester cleavable moieties.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphate diester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate diester linkage. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

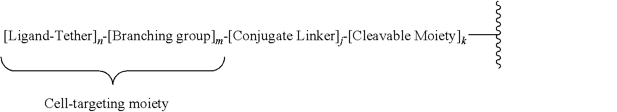

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphate diester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphate diester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphate diester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3 -di-O-methyl-D-mannopyranose, 2-deoxy-2-sulfoamino-D-glucopyranose and N-sulfo-D-glucosamine, and N-glycolyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-ghtco-heptopyranoside.

In certain embodiments, oligomeric compounds synthesized using processes described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO02012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Target Nucleic Acids

In certain embodiments, oligonucleotides synthesized using processes described herein comprise or consist of an oligonucleotide that is complementary to a target nucleic acid. In certain embodiments, the target nucleic acid is an endogenous RNA molecule. In certain embodiments, the target nucleic acid encodes a protein. In certain such embodiments, the target nucleic acid is an mRNA. In certain embodiments, an oligonucleotide is complementary to both a pre-mRNA and corresponding mRNA but only the mRNA is the target nucleic acid due to an absence of antisense activity upon hybridization to the pre-mRNA. In certain embodiments, an oligonucleotide is complementary to an exon-exon junction of a target mRNA and is not complementary to the corresponding pre-mRNA.

### Compound Isomers

Certain oligonucleotides synthesized using processes described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The oligonucleotides synthesized using processes described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S , ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine nucleobase could be described as a DNA having an RNA sugar, or as an RNA having a DNA nucleobase.

Accordingly, nucleic acid sequences provided herein, including, but not limited to those in the sequence listing, are intended to encompass nucleic acids containing any combination of unmodified or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein mC indicates a cytosine base comprising a methyl group at the 5-position.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Boronic Acid-Assisted Reversed-Phase Purification of Conjugated Oligonucleotides by HPLC

### Experimental Design

Oligomeric compounds and modified oligonucleotides were synthesized using standard procedures. Compound Nos. 304801 and 678354 have a sequence (from 5′ to 3′) of AGCTTCTTGTCCAGCTTTAT (SEQ ID NO: 1), and are 5-10-5 MOE gapmers having a central gap segment of ten 2′-β-D-deoxynucleosides flanked on each side by wing segments, each comprising five 2′-MOE modified nucleosides. Each internucleoside linkage is a phosphorothioate. All cytosine nucleobases throughout the modified oligonucleotides are 5-methylcytosines.

Compound No. 304801 is unconjugated. Compound No. 678354 has a GalNAc moiety conjugated to the 5′ oxygen of the oligonucleotide via a THA linker, as shown below:

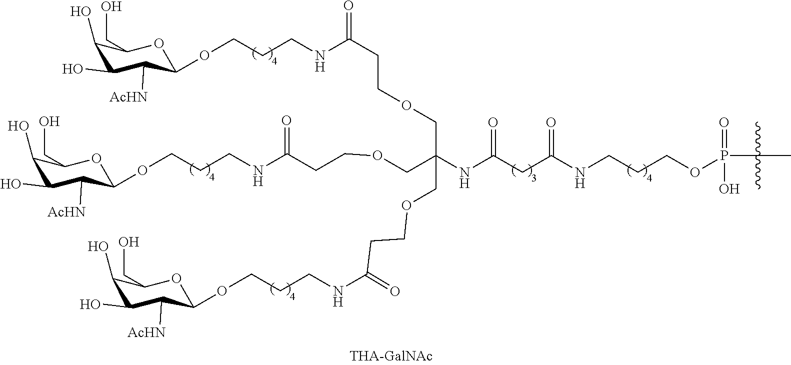

Separation of GalNAc-conjugated (304801) and unconjugated (678354) modified oligonucleotides was performed using RP-HPLC with mobile phases at different pH values and with or without phenylboronic acid (PBA), in order to determine improved conditions for the separation of conjugated and unconjugated oligonucleotides.

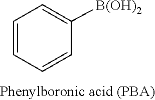

### Chromatographic Instrumentation and Conditions

RP-HPLC was performed on a Nanomicro Uni-PS 5-300 5 μm column (4.6×100 mm) using an Agilent 1220 Infinity LC. The column temperature was maintained at 32° C. The mobile phases for analytical chromatography were: (A) 10% MeOH, 0.2 M PBA, 0.2 M NaOAc, and (B) 70% MeOH, 0.2 M PBA, 0.2 M NaOAc, applied under the following conditions:

TABLE 1
Timetable for RP-HPLC gradient elution

| Time (min) | A (%) | B (%) |
|---|---|---|
| 0.0 | 100.0 | 0.0 |
| 0.8 | 100.0 | 0.0 |
| 5.3 | 0.0 | 100.0 |
| 7.3 | 0.0 | 100.0 |
| 7.4 | 100.0 | 0.0 |

Control conditions contained no PBA in either of the mobile phases, but followed the same gradient elution as listed in the table above. The injection sample contained a 10:3 ratio of GalNAc-conjugated (304801) to unconjugated (678354) modified oligonucleotides (10 mg/mL) in water. The injection volume was 20 μL, and the flow rate was 1000 μL/min. UV absorbance was monitored at 295 nm for phenyl boronic acid conditions and 254 nm and 295 nm for control conditions.

HPLC was carried out with mobile phases adjusted to pH values of 7, 8, 9, 10, 11, and 12, with and without PBA, yielding twelve chromatograms. The table below shows the retention times (RTs) of the GalNAc-conjugated modified oligonucleotide and unconjugated modified oligonucleotide compounds. A greater difference in retention time (ART) affords better separation of the conjugated and unconjugated modified oligonucleotides. The addition of phenyl boronic acid increased the separation of GalNAc-conjugated modified oligonucleotides from the contaminant unconjugated modified oligonucleotides at every pH tested. The greatest separation was observed at pH 8 with the addition of phenylboronic acid, while the greatest separation under standard conditions is achieved at pH 12.

TABLE 2
pH-dependent RP-HPLC retention times of conjugated and
unconjugated modified oligonucleotides

| | PBA | | | No PBA | | |
|---|---|---|---|---|---|---|
| | conjugated | unconjugated | Δ_{RT} | conjugated | unconjugated | Δ_{RT} |
| pH | RT (min) | RT (min) | (min) | RT (min) | RT (min) | (min) |
| 7 | 5.939 | 3.732 | 2.207 | 5.741 | 5.484 | 0.257 |
| 8 | 5.322 | 2.158 | 3.164 | 5.565 | 5.396 | 0.169 |
| 9 | 5.093 | 2.722 | 2.371 | 5.634 | 5.362 | 0.272 |
| 10 | 5.154 | 3.927 | 1.227 | 5.549 | 5.246 | 0.303 |
| 11 | 5.358 | 3.883 | 1.475 | 5.083 | 4.607 | 0.476 |
| 12 | 5.350 | 3.815 | 1.535 | 4.973 | 4.212 | 0.761 |

### Example 2: Effect of pH and Functional Group on Boronic Acid-Assisted Reversed-Phase Purification of Conjugated Oligonucleotides by HPLC

### Experimental Design

GalNAc-conjugated compound No. 678354 and its unconjugated counterpart 304801 (described herein above) were analyzed by RP-HPLC. RP-HPLC was run at pH 7, 8, 9, 10, 11, and 12 to determine the optimal pH for the largest retention time difference. Various boronic acids were evaluated for their effect on the retention times of the conjugated and unconjugated modified oligonucleotides. Twelve boronic acids shown below were assessed in RP-HPLC: phenylboronic acid (PBA), 4-fluorophenylboronic acid (4-FPBA), 4-(trifluoromethyl)phenylboronic acid (4-CF₃PBA), 2-(trifluoromethyl)phenylboronic acid (2-CF₃PBA), p-tolylboronic acid (4-MePBA), o-tolylboronic acid (2-MePBA), 4-methoxyphenylboronic acid (4-MeOPBA), borate, methylboronic acid (MeBA), propylboronic acid (n-PrBA), butylboronic acid (n-BuBA), and pentylboronic acid (n-PenBA). A control experiment containing no boronic acid was also conducted.

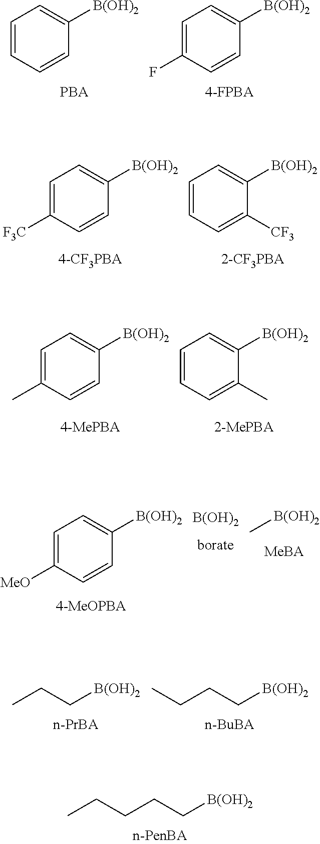

In addition, two immobilized boronic acid resins shown below were evaluated: Nanomicro Prototype C-1 5 μm column (4.6×100 mm) and Nanomicro Prototype D 5 μm column (4.6×100 mm).

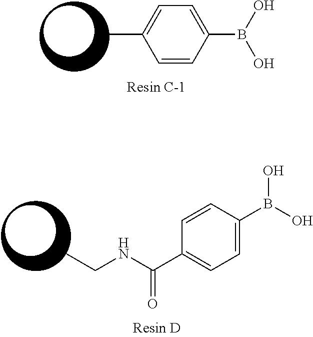

### Chromatographic Instrumentation and Conditions

RP-HPLC was performed using an Agilent 1220 Infinity LC. For the control conditions and the non-immobilized boronic acid resin conditions, the Nanomicro Uni-PS 5-300 5 μm column (4.6×100 mm) was used. The column temperature was maintained at 32° C. The mobile phases for analytical chromatography were: (A) 10% MeOH, 0-200 mM boronic acid, 0.2 M NaOAc, and (B) 70% MeOH, 0-200 mM boronic acid, 0.2 M NaOAc, where the concentration of each boronic acid was identical in mobile phase A and mobile phase B for a given HPLC run, applied under the following conditions:

TABLE 3
Timetable for RP-HPLC gradient elution

| Time (min) | A (%) | B (%) |
|---|---|---|
| 0.0 | 100.0 | 0.0 |
| 0.8 | 100.0 | 0.0 |
| 5.3 | 0.0 | 100.0 |
| 7.3 | 0.0 | 100.0 |
| 7.4 | 100.0 | 0.0 |

The concentration of boronic acid is listed in the tables below. The injection sample contained a 10:3 ratio of GalNAc-conjugated (678354) to unconjugated modified oligonucleotide (304801) (total concentration 10 mg/mL). The injection volume was 20 μL, and the flow rate was 1000 μL/min. UV absorbance was monitored at 295 nm.

The table below shows the difference in retention times (ART, minutes) of the GalNAc-conjugated modified oligonucleotide (678354) and the unconjugated modified oligonucleotide (304801). A greater difference in retention time (Δ_{RT}) affords better separation of the conjugated and unconjugated modified oligonucleotides. N.D. indicates conditions for which no data is available.

TABLE 4
Difference in retention times (minutes) of conjugated and
unconjugated modified oligonucleotides

| | Concen- | | | | | | |
|---|---|---|---|---|---|---|---|
| Boronic | tration | pH | | | | | |
| Acid | (mM) | 7 | 8 | 9 | 10 | 11 | 12 |
| control | N/A | 0.26 | 0.17 | 0.27 | 0.30 | 0.48 | 0.76 |
| PBA | 200 | 2.21 | 3.16 | 2.37 | 1.23 | 1.48 | 1.54 |
| 4-FPBA | 100 | N.D. | 1.43 | 0.95 | 1.08 | 1.19 | 1.29 |
| 4-CF₃PBA | 50 | N.D. | N.D. | 2.00 | 1.99 | 2.16 | 2.31 |
| 2-CF₃PBA | 200 | 1.26 | 2.80 | 3.03 | 3.13 | 4.06 | 4.40 |
| 4-MePBA | 50 | N.D. | N.D. | 1.33 | 1.58 | 1.58 | 1.75 |
| 2-MePBA | 200 | 2.04 | 2.35 | 3.54 | 2.16 | 1.83 | 2.09 |
| 4-MeOPBA | 50 | N.D. | 1.47 | 1.31 | 1.40 | 1.32 | 1.35 |
| borate | 200 | N.D. | 0.22 | 0.22 | 0.27 | 0.55 | 0.37 |
| MeBA | 200 | 0.30 | 0.32 | 0.32 | 0.34 | 0.57 | 0.75 |
| n-PrBA | 200 | N.D. | 0.65 | 0.68 | 0.76 | 0.86 | 0.00 |
| n-BuBA | 200 | 1.08 | 1.13 | 1.09 | 2.51 | 2.16 | 1.41 |
| n-PenBA | 50 | N.D. | 0.28 | 0.29 | 0.76 | 1.06 | 1.11 |
| Resin C-1 | N/A | 0.39 | 0.47 | 0.59 | 1.00 | 2.05 | 2.38 |
| Resin D | N/A | N.D. | N.D. | N.D. | N.D. | 1.02 | 1.50 |

The table below shows the resolution (Rₛ) of the GalNAc-conjugated oligonucleotide and unconjugated modified oligonucleotide compounds. Resolution is calculated from the retention times (t_{R}) and widths (W) of the conjugated and unconjugated modified oligonucleotide elution peaks as in the formula below:

$${R}_{S} = \frac{2 [ {( {t}_{R} )}_{conjugated} - {( {t}_{R} )}_{unconjugated} ]}{{W}_{conjugated} + {W}_{unconjugated}}$$

A higher resolution signifies better separation of the conjugated and unconjugated modified oligonucleotides. N.D. indicates conditions for which no data is available.

TABLE 5
Resolution of conjugated and unconjugated modified oligonucleotides

| | Concentration | pH | | | | | |
|---|---|---|---|---|---|---|---|
| Boronic Acid | (mM) | 7 | 8 | 9 | 10 | 11 | 12 |
| control | N/A | 1.05 | 1.07 | 1.14 | 1.26 | 1.38 | 3.47 |
| PBA | 200 | 8.10 | 11.79 | 7.43 | 5.97 | 7.07 | 7.17 |
| 4-FPBA | 100 | N.D. | 6.61 | 4.33 | 5.04 | 5.42 | 5.61 |
| 4-CF₃PBA | 50 | N.D. | N.D. | 6.29 | 7.01 | 7.41 | 5.30 |
| 2-CF₃PBA | 200 | 3.01 | 7.37 | 8.04 | 11.36 | 10.19 | 11.43 |
| 4-MePBA | 50 | N.D. | N.D. | 3.85 | 4.95 | 4.77 | 6.07 |
| 2-MePBA | 200 | 5.11 | 5.61 | 13.26 | 5.88 | 4.39 | 5.24 |
| 4-MeOPBA | 50 | N.D. | 6.04 | 4.74 | 5.12 | 5.09 | 3.29 |
| borate | 200 | N.D. | 0.81 | 0.83 | 0.97 | 1.75 | 1.55 |
| MeBA | 200 | 1.02 | 1.19 | 1.21 | 1.37 | 2.17 | 2.22 |
| n-PrBA | 200 | N.D. | 2.69 | 2.60 | 2.98 | 3.67 | 0.00 |
| n-BuBA | 200 | 3.23 | 3.53 | 3.50 | 8.05 | 6.71 | 4.21 |
| n-PenBA | 50 | N.D. | 1.16 | 1.37 | 3.50 | 4.22 | 3.11 |
| Resin C-1 | N/A | 0.42 | 0.64 | 0.74 | 1.01 | 1.29 | 1.45 |
| Resin D | N/A | N.D. | N.D. | N.D. | N.D. | 1.00 | 1.04 |

### Example 3: Boronic Acid-Assisted Reversed-Phase Purification of Conjugated Oligonucleotides by HPLC Involving a Washing Mobile Phase

### Experimental Design

Oligomeric compounds and modified oligonucleotides were synthesized using standard procedures. Compound Nos. 700994 and 682884 have a sequence (from 5′ to 3′) of TCTTGGTTACATGAAATCCC (SEQ ID NO: 2), and are 5-10-5 MOE gapmers having a central gap segment often 2′-β-D-deoxynucleosides flanked on each side by wing segments, each comprising five 2′-MOE modified nucleosides. The internucleoside linkage motif for the gapmers is (from 5′ to 3′): soooossssssssssooss; wherein each ‘o’ represents a phosphodiester internucleoside linkage and each ‘s’ represents a phosphorothioate internucleoside linkage. All cytosine nucleobases throughout the modified oligonucleotides are 5-methylcytosines.

Compound No. 700994 is unconjugated. Compound No. 682884 has a GalNAc moiety conjugated to the 5′ oxygen of the oligonucleotide via a THA linker, as shown below:

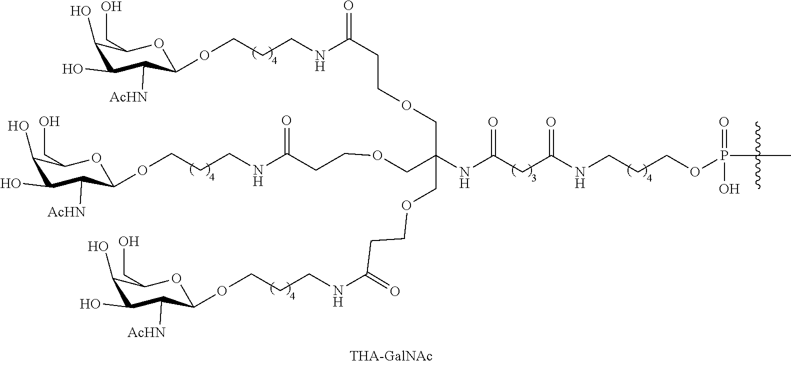

Separation of GalNAc-conjugated (682884) and unconjugated (700994) modified oligonucleotides was performed using RP-HPLC with a three mobile phase system in which phenyl boronic acid was included in only the equilibration solution.

### Chromatographic Instrumentation and Conditions

RP-HPLC was performed using an ÄKTA Avant 25 system using a Nanomicro Uni-PS 30-300 30 μm column (10×100 mm). The mobile phases for chromatography were: (A) 10% MeOH, 0.2 M NaOAc, 0.2 M PBA at pH=8, (B) 10% MeOH, 0.2 M NaOAc at pH=12, (C) 70% MeOH, 0.2 M NaOAc at pH=8, or (D) 10-100% MeOH, 0.2 M NaOAc at pH=12. The mobile phase was applied in either a linear gradient involving components A, B, and C as described in Table 6, or in a step gradient involving components A, B, and D as described in Table 7.

TABLE 6
Timetable for RP-HPLC linear gradient
elution of ternary mobile phase

| Time (min) | A (%) | B (%) | C (%) |
|---|---|---|---|
| 0.0 | 100.0 | 0.0 | 0.0 |
| 15.7 | 100.0 | 0.0 | 0.0 |
| 55.0 | 0 | 100.0 | 0.0 |
| 94.3 | 0 | 0.0 | 100.0 |

TABLE 7
Timetable for RP-HPLC step gradient
elution of ternary mobile phase

| Time (min) | A (%) | B (%) | D (%) |
|---|---|---|---|
| 0.0 | 100.0 | 0.0 | 0.0 |
| 15.7 | 100.0 | 0.0 | 0.0 |
| 15.71 | 0 | 100.0 | 0.0 |
| 58.9 | 0 | 100.0 | 0.0 |
| 58.91 | 0 | 0 | 100.0 |
| 74.6 | 0 | 0.0 | 100.0 |

The injection sample contained crude mixture of GalNAc-conjugated oligonucleotide (Compound No. 682884, described herein above) and impurities including unconjugated oligonucleotide sequences (Compound No. 700994, described herein above) and synthesis-related small molecules. The injection volume was 8.0 mL at 21.5 mg/mL of the impure mixture, and the flow rate was 2.0 mL/min. UV absorbance was monitored at 295, 260, and 220 nm.

The linear gradient process improved the purity of the GalNAc-conjugated oligonucleotide from 70% to 92%. The step gradient process improved the purity of the GalNAc-conjugated oligonucleotide from 70% to 90%.

What is claimed:

1. A process for separating a designated oligomeric compound from at least one contaminant in sample solution,
wherein the designated oligomeric compound comprises a modified oligonucleotide and a conjugate group having at least one carbohydrate moiety,
comprising:
g) Providing an HPLC column packed with an HPLC stationary phase,
h) Adding equilibration solution to the HPLC column to equilibrate the column,
i) Adding the designated oligomeric compound and at least one contaminant in sample solution onto the equilibrated HPLC column,
j) Adding equilibration solution to the HPLC column,
k) Optionally, washing the HPLC column with washing solution,
l) Adding elution solution to the HPLC column to elute the oligomeric compound from the column, and collecting the eluate in separate tubes at various time points, and
thereby separating the designated oligomeric compound from the contaminant;
wherein:
the equilibration solution comprises 0% to 20% organic solvent, 50 mM to 500 mM salt, and 50 to 200 mM of a boronic acid, and has a pH of 7.0 or greater;
the washing solution comprises 0% to 50% of the same organic solvent as the equilibration solution, and 50 to 500mM salt, and has a pH of 10 or greater; and
the elution solution comprises 50% to 90% of the same organic solvent as is in the equilibration solution, 50 to 500 mM salt, and 0-200 mM of a boronic acid, and has a pH of 7.0 or greater.

2. The process of claim 1, wherein at least one column volume of equilibration solution is added to the HPLC column in step (d).

3. The process of claim 1 or 2 that does not include washing step (e).

4. The process of claim 1 or 2 comprising the washing step (e).

5. The process of claim 4, wherein at least 2 column volumes of washing solution are added.

6. The process of any of claims 1-5, wherein at least one column volume of elution solution is added at step (f).

7. The process of claim 1, wherein the boronic acid of the equilibration solution is selected from phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, 4-methoxyphenylboronic acid, boric acid, methylboronic acid, propylboronic acid, butylboronic acid, and pentylboronic acid.

8. The process of claim 7, wherein the boronic acid is an aryl boronic acid.

9. The process of claim 7, wherein the aryl boronic acid is selected from phenyl boronic acid, 4-fluorophenyl boronic acid, 4-(trifluoromethyl)phenylboronic acid, 2-(trifluoromethyl)phenylboronic acid, p-tolylboronic acid, o-tolylboronic acid, and 4-methoxyphenylboronic acid.

10. The process of claim 7, wherein the boronic acid is selected from butyl boronic acid, propyl boronic acid, and pentyl boronic acid.

11. The process of any of claims 1-10, wherein the equilibration solution comprises 50 mM of the boronic acid.

12. The process of any of claims 1-10, wherein the equilibration solution comprises 100 mM of the boronic acid.

13. The process of any of claims 1-10, wherein the equilibration solution comprises 200 mM of the boronic acid.

14. The process of any of claims 1-13, wherein the pH of the equilibration solution is from 7 to 7.5.

15. The process of any of claims 1-13, wherein the pH of the equilibration solution is from 7.5 to 8.5.

16. The process of any of claims 1-13, wherein the pH of the equilibration solution is from 8.5 to 9.5.

17. The process of any of claims 1-13, wherein the pH of the equilibration solution is from 9.5 to 10.5.

18. The process of any of claims 1-13, wherein the pH of the equilibration solution is from 10.5 to 11.5.

19. The process of any of claims 1-13, wherein the pH of the equilibration solution is from 11.5 to 12.5.

20. The process of any of claims 1-19, wherein the elution solution contains 50 mM of the boronic acid.

21. The process of any of claims 1-19, wherein the elution solution contains 100 mM of the boronic acid.

22. The process of any of claims 1-19, wherein the elution solution contains 150 mM of the boronic acid.

23. The process of any of claims 1-19, wherein the elution solution contains 200 mM of the boronic acid.

24. The process of any of claims 1-23, wherein the pH of the elution solution is from 7 to 7.5.

25. The process of any of claims 1-23, wherein the pH of the elution solution is from 7.5 to 8.5.

26. The process of any of claims 1-23, wherein the pH of the elution solution is from 8.5 to 9.5.

27. The process of any of claims 1-23, wherein the pH of the elution solution is from 9.5 to 10.5.

28. The process of any of claims 1-23, wherein the pH of the elution solution is from 10.5 to 11.5.

29. The process of any of claims 1-23, wherein the pH of the elution solution is from 11.5 to 12.5.

30. The process of any of claims 1-29, wherein the equilibration solution and the elution solution have the same concentration of boronic acid.

31. The process of any of claims 1-30, wherein the equilibration solution and the elution solution have the same pH.

32. The process of any of claims 1-31, wherein the equilibration solution and the elution solution are the same other than the organic solvent:water ratio.

33. The process of any of claims 1-32, wherein the equilibration solution contains 3-8%, 8-12%, 12-18%, 18-22%, 22-27%, or 27-33% organic solvent.

34. The process of any of claims 1-33, wherein the elution solution contains 55-65%, 65-75%, or 75-85% organic solvent.

35. The process of claim 33 or 34, wherein the equilibration solution comprises 10% organic solvent and the elution solution contains 70% organic solvent.

36. The process of any of claims 4-35, wherein the washing solution contains 3-8%, 8-12%, or 12-18% organic solvent.

37. The process of any of claims 1-36, wherein the organic solvent is selected from acetonitrile, methanol, isopropanol, ethanol, and tetrahydrofuran.

38. The process of claim 37, wherein the organic solvent is methanol.

39. The process of any of claims 1-38, wherein the equilibration solution and the elution solution contain 150-250 mM salt.

40. The process of claims 39, wherein the salt of the equilibration solution and the elution solution is, independently, sodium acetate, sodium chloride, sodium carbonate, or sodium phosphate.

41. The process of claim 40, wherein the salt is sodium acetate.

42. The process of any of claims 1-41, wherein the elution solution is added in a step gradient.

43. The process of any of claims 1-42, wherein the elution solution is added in a linear gradient.

44. The process of any of claim 1-2 or 4-43, wherein the washing solution is added in a step gradient.

45. The process of any of claim 1-2 or 4-43, wherein the washing solution is added in a linear gradient.

46. The process of any of claims 1-45, wherein the HPLC stationary phase is selected from polystyrene, surface-modified polystyrene, surface-modified silica, and surface-modified methylacrylate.

47. The process of any of claims 1-46, wherein the HPLC stationary phase is monodisperse polystyrene.

48. The process of any of claims 1-46, wherein the HPLC stationary phase is surface-modified and the surface-modification comprises a boronic acid functionality.

49. The process of any of claims 1-48, wherein the designated oligomeric compound comprises a modified oligonucleotide consisting of 10-30 linked nucleosides.

50. The process of any of claims 1-49, wherein the designated oligomeric compound comprises a conjugate group having at least one GalNAc sugar.

51. The process of claim 50, wherein the conjugate group comprises a triantennary GalNAc cell-targeting moiety.

52. The process of claim 51, wherein the conjugate group comprises the structure:

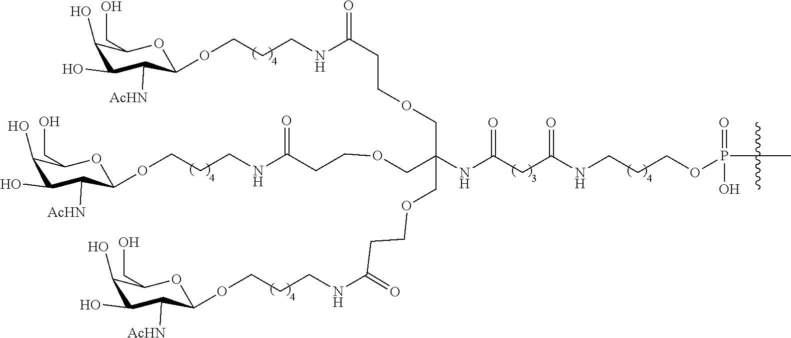

53. The process of any of claims 1-52, wherein at least one contaminant is an unconjugated modified oligonucleotide consisting of 10-30 linked nucleosides.

54. The process of claim 53, wherein the unconjugated modified oligonucleotide has the same nucleobase sequence, internucleoside linkage chemistry, and sugar motif as the modified oligonucleotide of the designated oligomeric compound.
